(19) United States
(12) Patent Application Publication
(10) Pub. No.: US 2026/0096844 A1
(43) Pub. Date: Apr. 9, 2026

(54) SPRING ASSISTED SEALING INSTRUMENT

(71) Applicant: Covidien LP, Mansfield, MA (US)

(72) Inventors: Dylan R. Kingsley, Minneapolis, MN (US); Jason G. Weihe, Minneapolis, MN (US); Christopher T. Tschudy, Minneapolis, MN (US); Brock Kopp, Minneapolis, MN (US); Zachary S. Heiliger, Minneapolis, MN (US); Russell W. Holbrook, Minneapolis, MN (US); William R. Whitney, Minneapolis, MN (US); Daniel A. Joseph, Minneapolis, MN (US)

(73) Assignee: Covidien LP, Mansfield, MA (US)

(21) Appl. No.: 19/115,395

(22) PCT Filed: Sep. 26, 2023
(86) PCT No.: PCT/IB2023/059505

§ 371 Date: Mar. 26, 2025

Related U.S. Application Data
(60) Provisional application No. 63/411,696, filed on Sep. 30, 2022.

(51) Int. Cl. A61B 18/14; A61B 34/30; A61B 90/00

(52) U.S. Cl. CPC A61B 18/1442; A61B 34/30; A61B 90/03; A61B 2018/1455; A61B 2090/034

(57) ABSTRACT

A surgical instrument includes a housing having a shaft extending therefrom with an end effector at a distal end thereof. The end effector includes first and second jaw members moveable between a spaced apart configuration and a closed position to grasp tissue between jaw members. A drive assembly including a drive rod is associated with the movable jaw member. A spring having a spring rate “k” is operably associated with the drive rod and is configured to offload forces associated therewith during actuation thereof. The spring includes a first length wherein adjacent coils thereof are spaced a distance relative to one another such that the spring offloads forces associated with the drive rod as per the spring rate “k” of the spring and a fully compressed length wherein the adjacent coils of the spring abut one another and the forces associated with the drive rod are transferred through the spring.

### FIELD

The present disclosure relates to surgical instruments and, more specifically, to sealing instruments such as, for example, for use in robotic surgical systems, and methods relating to the same.

### BACKGROUND

Surgical systems are increasingly utilized in various different surgical procedures. For example, robotic surgical systems include a console supporting a robotic arm. One or more different surgical instruments may be configured for use with the robotic surgical system and selectively mountable to the robotic arm. The robotic arm provides one or more inputs to the mounted surgical instrument to enable operation of the mounted surgical instrument.

When treating tissue, the closure force and sealing force between jaw members of a surgical instrument may need to be controlled to properly treat tissue and avoid tissue and/or instrument damage. As a result, instrument manufacturers typically need to include ways to monitor the forces on the tissue or other aspects of the surgical instrument during treatment.

### SUMMARY

As used herein, the term “distal” refers to the portion that is being described which is further from an operator (whether a human surgeon or a surgical robot), while the term “proximal” refers to the portion that is being described which is closer to the operator. The terms “about,” substantially,” and the like, as utilized herein, are meant to account for manufacturing, material, environmental, use, and/or measurement tolerances and variations. Further, to the extent consistent, any of the aspects described herein may be used in conjunction with any or all of the other aspects described herein. Moreover, rotation may be measure in degrees or radians.

Provided in accordance with aspects of the present disclosure is a surgical instrument including a housing having a shaft extending therefrom including an end effector at a distal end thereof, the end effector including first and second jaw members. One or both of the jaw members is moveable between a spaced apart configuration relative to the other jaw member and a closed position wherein the jaw members cooperate to grasp tissue therebetween. A drive assembly including a drive rod is operably associated with the one or more jaw members and is actuatable to move the one or more jaw members between the spaced apart position and the closed position. A spring having a spring rate “k” is operably associated with the drive rod and is configured to offload forces associated therewith during actuation of the drive rod. The spring includes a first length wherein adjacent coils of the spring are spaced a distance relative to one another such that the spring offloads forces associated with the drive rod as per the spring rate “k” of the spring and a fully compressed length wherein the adjacent coils of the spring abut one another and the forces associated with the drive rod are transferred through the spring.

In aspects according to the present disclosure, the spring is configured to offload a certain percentage of forces associated to the drive rod prior to reaching a fully compressed length. In other aspects according to the present disclosure, the percentage of forces offloaded to the drive rod is in the range of about 30% to about 80%.

In aspects according to the present disclosure, in the fully compressed length, forces from an input operably associated with the drive assembly are transferred directly to the drive rod. In other aspects according to the present disclosure, the input is operably associated with the drive assembly and includes an input coupler from a robotic surgical system. In yet other aspects according to the present disclosure, the input operably associated with the drive assembly includes a movable handle extending from the housing.

Provided in accordance with aspects of the present disclosure is a surgical instrument including a housing having a shaft extending therefrom including an end effector at a distal end thereof, the end effector including first and second jaw members. A drive assembly is included having: a drive rod operably associated with the one or more jaw members and is actuatable to move the one or more jaw members between the spaced apart position and the closed position; and a progressive spring is operably associated with the drive rod and includes a variable spring rate “k” configured to offload forces associated with the drive rod during actuation thereof, the spring including a first spring rate “k₁” associated with initial activation thereof and one or more different spring rates “k₂” as the spring is compressed.

In aspects according to the present disclosure, the first spring rate “k₁” is less than the at least one different spring rate “k₂”.

In aspects according to the present disclosure, a diameter of the progressive spring varies along a length thereof. In other aspects according to the present disclosure, the spring rate of the progressive spring is directly proportional to the diameter thereof.

In aspects according to the present disclosure, the spring rate of the progressive spring is non-linear.

Provided in accordance with aspects of the present disclosure is a surgical instrument including a housing having a shaft extending therefrom an end effector at a distal end thereof, the end effector including first and second jaw members. One or both of the jaw members is moveable between a spaced apart configuration relative to the other jaw member and a closed position wherein the jaw members cooperate to grasp tissue therebetween. A drive assembly is included having a drive rod operably associated with the one or more jaw members and is actuatable to move the one or more jaw members between the spaced apart position and the closed position for grasping tissue.

A knife tube is disposed within the shaft, the knife tube having a knife at a distal end thereof configured to cut tissue disposed between the jaw members. A spring having a spring rate “K” is operably associated with the knife tube and is configured to offload forces associated therewith during actuation of the knife tube. The spring includes a first length wherein adjacent coils of the spring are spaced a distance relative to one another such that the spring offloads forces associated with the knife tube as per the spring rate “k” of the spring and a fully compressed length wherein the adjacent coils of the spring abut one another and the forces associated with the knife tube are transferred through the spring to prevent damage to the knife.

In aspects according to the present disclosure, the spring is configured to offload a certain percentage of forces associated to the knife tube prior to reaching a fully compressed length.

In aspects according to the present disclosure, an input coupler from a robotic surgical system is configured to actuate the knife tube. In other aspects according to the present disclosure, a trigger is operably associated with the knife tube and is configured to actuate the knife tube upon movement thereof.

In aspects according to the present disclosure, the instrument further includes a second spring operably associated with the knife tube, the second spring configured to offload forces associated with articulation of the shaft and the knife tube disposed therein.

### BRIEF DESCRIPTION OF THE DRAWINGS

Various aspects and features of the present disclosure are described hereinbelow with reference to the drawings wherein like numerals designate identical or corresponding elements in each of the several views.

FIG. 1A is a perspective view of a robotic surgical instrument provided in accordance with the present disclosure configured for mounting on a robotic arm of a robotic surgical system;

FIG. 1B perspective view of an endoscopic surgical instrument provided in accordance with the present disclosure;

FIG. 2A is a front, perspective view of a proximal portion of the surgical instrument of FIG. 1A with an outer shell removed;

FIG. 2B is a rear, perspective view of the proximal portion of the surgical instrument of FIG. 1A with the outer shell removed;

FIG. 3 is a front, perspective view of the proximal portion of the surgical instrument of FIG. 1A with the outer shell and additional internal components removed;

FIG. 4 is a schematic illustration of an exemplary robotic surgical system configured to releasably receive the surgical instrument of FIG. 1A;

FIG. 5 is a front, perspective view of a jaw drive sub-assembly of the surgical instrument of FIG. 1A;

FIG. 6 is a rear, perspective view of the jaw drive sub-assembly of the surgical instrument of FIG. 1A;

FIG. 7 is an exploded, perspective view of the jaw drive sub-assembly of the surgical instrument of FIG. 1A;

FIG. 8 is a perspective view of a distal potion of the surgical instrument of FIG. 1A with the end effector assembly disposed in an open position;

FIG. 9 is a longitudinal, cross-sectional view of a proximal portion of the surgical instrument of FIG. 1A illustrating the jaw drive sub-assembly transitioning the end effector assembly from the open position towards a closed position;

FIG. 10 is a perspective view of the distal potion of the surgical instrument of FIG. 1A with the end effector assembly disposed in the closed position;

FIG. 11 is a longitudinal, cross-sectional view of the proximal portion of the surgical instrument of FIG. 1A illustrating the jaw drive sub-assembly retaining the end effector assembly in the closed position;

FIGS. 12A-12C are enlarged views of a spring in accordance with one embodiment of the present disclosure for use with the surgical instruments of FIGS. 1A and 1B;

FIGS. 13A and 13B are enlarged views of a linear spring and a progressive spring, respectively, in accordance with another embodiment of the present disclosure for use with the surgical instruments of FIGS. 1A and 1B;

FIG. 13C is a graph showing the respective various springs rates for the springs illustrated in FIGS. 13A and 13B; and

FIGS. 14A and 14B are enlarged views of a knife tube for use with a load cell or one or more springs to prevent damage to the knife tube in accordance with another embodiment of the present disclosure.

### DETAILED DESCRIPTION

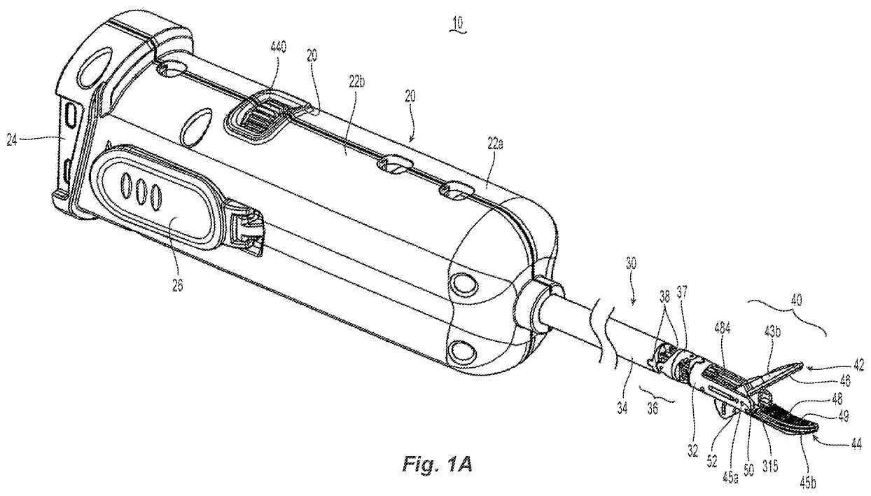

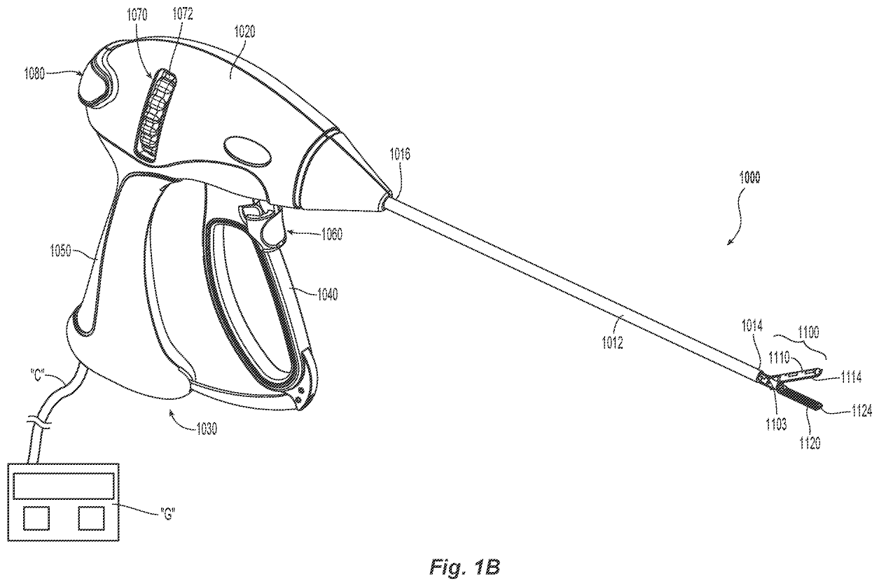

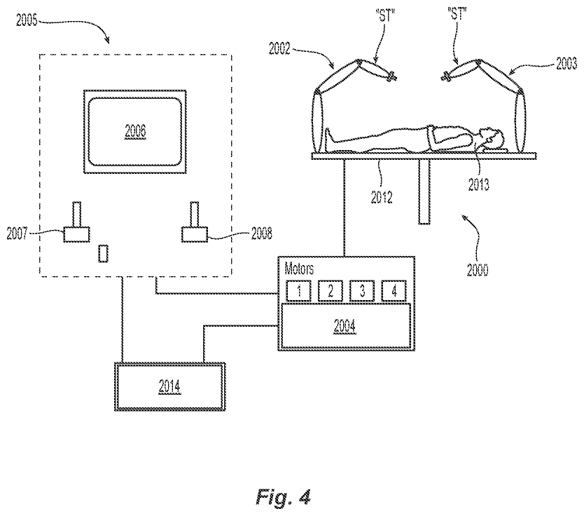

Referring to FIG. 1A, and 2A-3, surgical instrument 10 provided in accordance with the present disclosure generally includes a housing 20, a shaft 30 extending distally from housing 20, an end effector assembly 40 extending distally from shaft 30, and an actuation assembly 100 disposed within housing 20 and operably associated with end effector assembly 40. Instrument 10 is detailed herein as an articulating electrosurgical forceps configured for use with a robotic surgical system, e.g., robotic surgical system 2000 (FIG. 4). However, the aspects and features of instrument 10 provided in accordance with the present disclosure, detailed below, are equally applicable for use with other suitable surgical instruments, e.g., graspers, staplers, clip appliers, and/or in other suitable surgical systems, e.g., motorized or other power-driven systems.

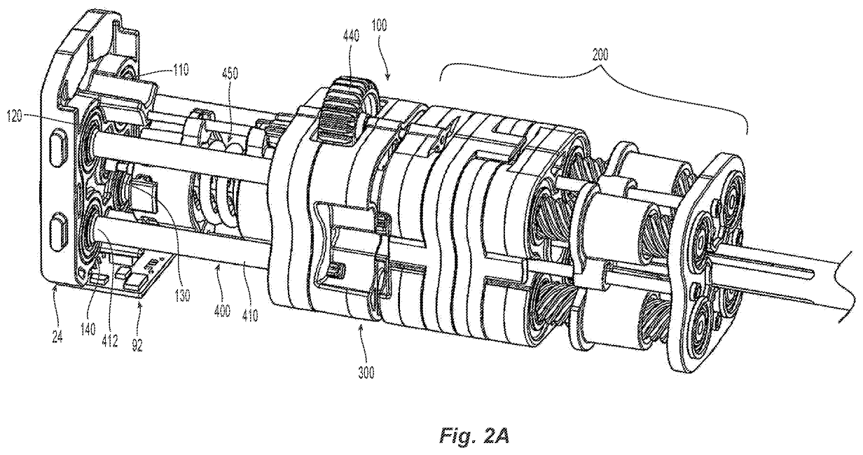

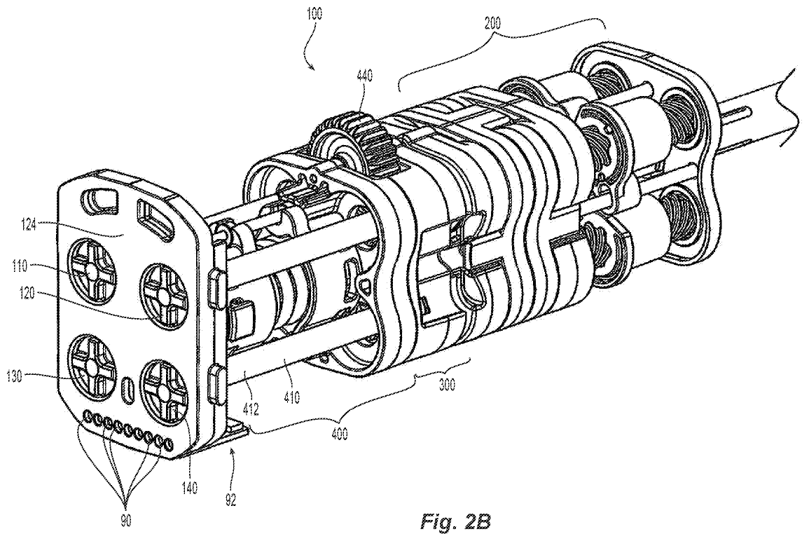

With particular reference to FIG. 1A, housing 20 of instrument 10 includes first and second body portion 22a, 22b and a proximal face plate 24 that cooperate to enclose actuation assembly 100 therein. Proximal face plate 24 includes apertures defined therein through which input couplers 110-140 (FIG. 2B) of actuation assembly 100 extend. A pair of latch levers 26 (only one of which is illustrated in FIG. 1A) extending outwardly from opposing sides of housing 20 enable releasable engagement of housing 20 with a robotic arm of a surgical system, e.g., robotic surgical system 2000 (FIG. 4). An aperture 28 defined through housing 20 permits thumbwheel 440 to extend therethrough to enable manual manipulation of thumbwheel 440 from the exterior of housing 20 to permit manual opening and closing of end effector assembly 40.

Referring to FIG. 1B, an endoscopic electrosurgical forceps provided in accordance with the present disclosure is shown generally identified by reference numeral 1000. Aspects and features of forceps 10 not germane to the understanding of the present disclosure are omitted to avoid obscuring the aspects and features of the present disclosure in unnecessary detail.

Forceps 1000 includes a housing 1020, a handle assembly 1030, a trigger assembly 1060, a rotating assembly 1070, an activation switch 1080, and an end effector assembly 1100. Forceps 1010 further includes a shaft 1012 having a distal end portion 1014 configured to (directly or indirectly) engage end effector assembly 1100 and a proximal end portion 1016 that (directly or indirectly) engages housing 1020. Forceps 1000 also includes cable “C” that connects forceps 1000 to an energy source, e.g., an electrosurgical generator “G.” Cable “C” includes a wire (or wires) (not shown) extending therethrough that has sufficient length to extend through shaft 1012 in order to connect to one or both tissue-treating surfaces 1114, 1124 of jaw members 1110, 1120, respectively, of end effector assembly 1100 to provide energy thereto. Activation switch 1080 is coupled to tissue-treating surfaces 1114, 1124 and the electrosurgical generator “G” for enabling the selective activation of the supply of energy to jaw members 1110, 1120 for treating, e.g., cauterizing, coagulating/desiccating, and/or sealing, tissue.

Handle assembly 1030 of forceps 1000 includes a fixed handle 1050 and a movable handle 1040. Fixed handle 1050 is integrally associated with housing 1020 and handle 1040 is movable relative to fixed handle 1050. Movable handle 1040 of handle assembly 1030 is operably coupled to a drive assembly (not shown) that, together, mechanically cooperate to impart movement of one or both of jaw members 1110, 1120 of end effector assembly 1100 about a pivot 1103 between a spaced-apart position and an approximated position to grasp tissue between tissue-treating surfaces 1114, 1124 of jaw members 1110, 1120. As shown in FIG. 1B, movable handle 1040 is initially spaced-apart from fixed handle 1050 and, correspondingly, jaw members 1110, 1120 of end effector assembly 1100 are disposed in the spaced-apart position. Movable handle 1040 is depressible from this initial position to a depressed position corresponding to the approximated position of jaw members 1110, 1120. Rotating assembly 1070 includes a rotation wheel 1072 that is selectively rotatable in either direction to correspondingly rotate end effector assembly 1100 relative to housing 1020.

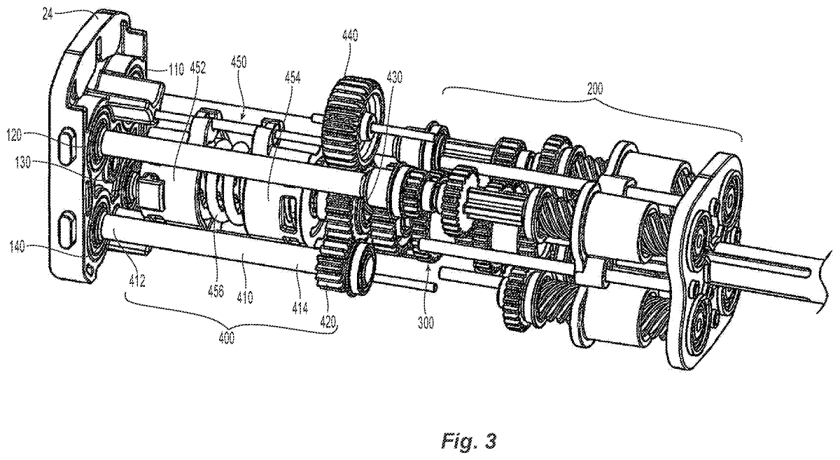

Referring back to FIGS. 2A-3, a plurality of electrical contacts 90 extend through one or more apertures defined through proximal face plate 24 to enable electrical communication between instrument 10 and robotic surgical system 2000 (FIG. 4) when instrument 10 is engaged thereon, e.g., for the communication of data, control, and/or power signals therebetween. As an alternative to electrical contacts 90 extending through proximal face plate 24, other suitable transmitter, receiver, and/or transceiver components to enable the communication of data, control, and/or power signals are also contemplated, e.g., using RFID, Bluetooth®, WiFi®, or via any other suitable wired, wireless, contacted, or contactless communication method. At least some of the electrical contacts 90 are electrically coupled with electronics 92 mounted on an interior side of proximal face plate 24, e.g., within housing 20. Electronics 92 may include, for example, a storage device, a communications device (including suitable input/output components), and a CPU including a memory and a processor. Electronics 92 may be mounted on a circuit board or otherwise configured, e.g., as a chip.

The storage device of electronics 92 stores information relating to surgical instrument such as, for example: the item number, e.g., SKU number; date of manufacture; manufacture location, e.g., location code; serial number; lot number; use information; setting information; adjustment information; calibration information; security information, e.g., encryption key(s), and/or other suitable additional or alternative data. The storage device of electronics 92 may be, for example, a magnetic disk, flash memory, optical disk, or other suitable data storage device.

As an alternative or in addition to storing the above-noted information in the storage device of electronics 92, some or all of such information, e.g., the use information, calibration information, setting information, and/or adjustment information, may be stored in a storage device associated with robotic surgical system 2000 (FIG. 4), a remote server, a cloud server, etc., and accessible via instrument 10 and/or robotic surgical system 2000 (FIG. 4). In such configurations, the information may, for example, be updated by manufacturer-provided updates, and/or may be applied to individual instruments, units of instruments (e.g., units from the same manufacturing location, manufacturing period, lot number, etc.), or across all instruments. Further still, even where the information is stored locally on each instrument, this information may be updated by manufacturer-provided updates manually or automatically upon connection to the robotic surgical system 2000 (FIG. 4).

Referring again to FIG. 1A, shaft 30 of instrument 10 includes a distal segment 32, a proximal segment 34, and an articulating section 36 disposed between the distal and proximal segments 32, 34, respectively. Articulating section 36 includes one or more articulating components 37, e.g., links, joints, etc. A plurality of articulation cables 38, e.g., four (4) articulation cables, or other suitable actuators, extend through articulating section 36. More specifically, articulation cables 38 are operably coupled to distal segment 32 of shaft 30 at the distal ends thereof and extend proximally from distal segment 32 of shaft 30, through articulating section 36 of shaft 30 and proximal segment 34 of shaft 30, and into housing 20, wherein articulation cables 38 operably couple with an articulation sub-assembly 200 of actuation assembly 100 to enable selective articulation of distal segment 32 (and, thus end effector assembly 40) relative to proximal segment 34 and housing 20, e.g., about at least two axes of articulation (yaw and pitch articulation, for example). Articulation cables 38 are arranged in a generally rectangular configuration, although other suitable configurations are also contemplated. In some configurations, as an alternative, shaft 30 is substantially rigid, malleable, or flexible and not configured for active articulation.

With respect to articulation of end effector assembly 40 relative to proximal segment 34 of shaft 30, actuation of articulation cables 38 may be accomplished in pairs. More specifically, in order to pitch end effector assembly 40, the upper pair of cables 38 are actuated in a similar manner while the lower pair of cables 38 are actuated in a similar manner relative to one another but an opposite manner relative to the upper pair of cables 38. With respect to yaw articulation, the right pair of cables 38 are actuated in a similar manner while the left pair of cables 38 are actuated in a similar manner relative to one another but an opposite manner relative to the right pair of cables 38. Other configurations of articulation cables 38 or other articulation actuators are also contemplated.

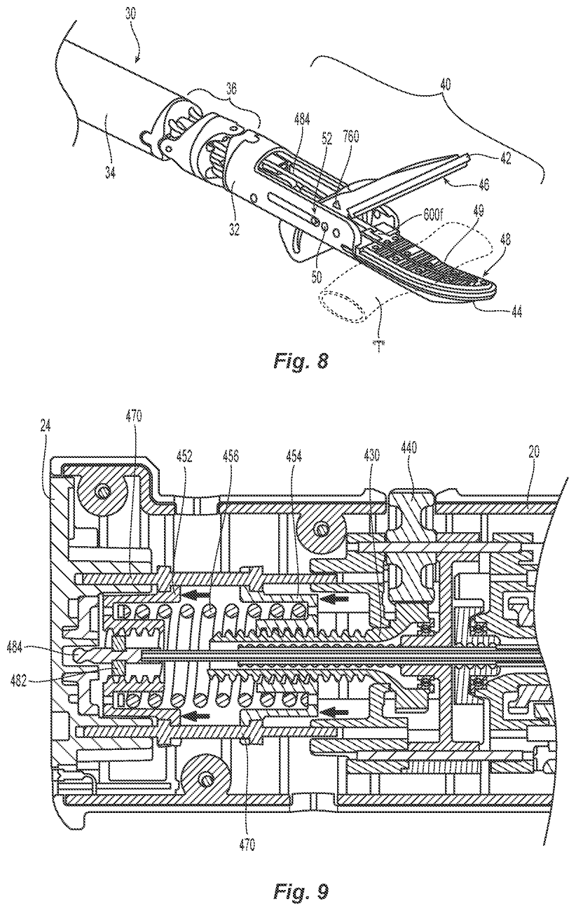

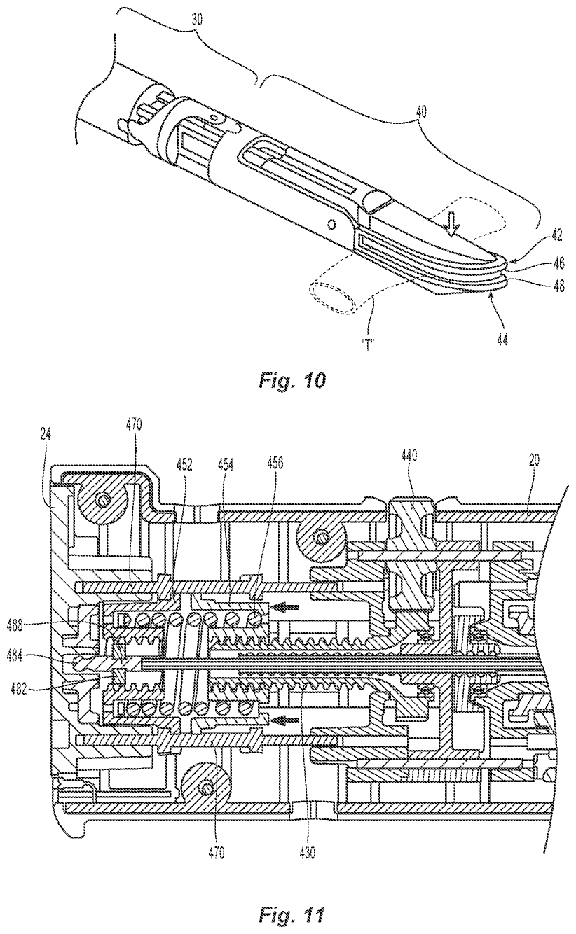

Continuing with reference to FIG. 1A, end effector assembly 40 includes first and second jaw members 42, 44, respectively. Each jaw member 42, 44 includes a proximal flange portion 43a, 45a and a distal body portion 43b, 45b, respectively. Distal body portions 43b, 45b define opposed tissue-contacting surfaces 46, 48, respectively. Proximal flange portions 43a, 45a are pivotably coupled to one another about a pivot 50 and are operably coupled to one another via a cam-slot assembly 52 including a cam pin slidably received within cam slots defined within the proximal flange portion 43a, 45a of at least one of the jaw members 42, 44, respectively, to enable pivoting of jaw member 42 relative to jaw member 44 and distal segment 32 of shaft 30 between a spaced-apart position (e.g., an open position of end effector assembly 40) and an approximated position (e.g., a closed position of end effector assembly 40) for grasping tissue “T” (FIGS. 8 and 10) between tissue-contacting surfaces 46, 48. As an alternative to this unilateral configuration, a bilateral configuration may be provided whereby both jaw members 42, 44 are pivotable relative to one another and distal segment 32 of shaft 30. Other suitable jaw actuation mechanisms are also contemplated.

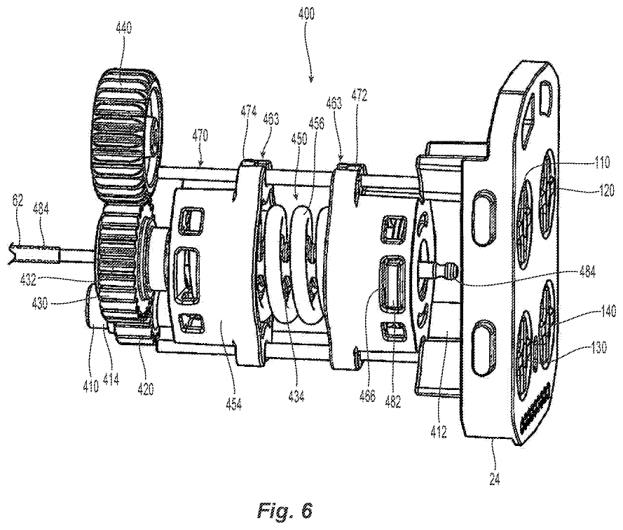

In configurations, a longitudinally-extending knife channel 49 (only knife channel 49 of jaw member 44 is illustrated; the knife channel of jaw member 42 is similarly configured) is defined through the tissue-contacting surface 46, 48 of one or both jaw members 42, 44. In such embodiments, a knife assembly including a knife tube 62 (FIG. 6) extending from housing 20 through shaft 30 to end effector assembly 40 and a knife blade 315 disposed within end effector assembly 40 between jaw members 42, 44 is provided. The knife blade 315 is selectively translatable through the knife channel(s) 49 and between the jaw member 42, 44 to cut tissue “T” (FIGS. 8 and 10) grasped between tissue-contacting surfaces 46, 48 of jaw members 42, 44, respectively. The knife tube 62 is operably coupled to a knife drive sub-assembly 300 (FIG. 3) of actuation assembly 100 (FIGS. 2A-2B) at a proximal end thereof to enable the selective actuation of the knife tube 62 to, in turn, reciprocate the knife blade 315 between jaw members 42, 44 to cut tissue “T” (FIGS. 8 and 10) grasped between tissue-contacting surfaces 46, 48. As an alternative to a longitudinally-advanceable mechanical knife, other suitable mechanical cutters are also contemplated, e.g., guillotine-style cutters, as are energy-based cutters, e.g., RF electrical cutters, ultrasonic cutters, etc., in static or dynamic configurations.

Referring still to FIG. 1A, a drive rod 484 is operably coupled to cam-slot assembly 52 of end effector assembly 40, e.g., engaged with the cam pin thereof, such that longitudinal actuation of drive rod 484 pivots jaw member 42 relative to jaw member 44 between the spaced-apart and approximated positions. More specifically, urging drive rod 484 proximally pivots jaw member 42 relative to jaw member 44 towards the approximated position while urging drive rod 484 distally pivots jaw member 42 relative to jaw member 44 towards the spaced-apart position. However, other suitable mechanisms and/or configurations for pivoting jaw member 42 relative to jaw member 44 between the spaced-apart and approximated positions in response to selective actuation of drive rod 484 are also contemplated. Drive rod 484 extends proximally from end effector assembly 40 through shaft 30 and into housing 20 wherein drive rod 484 is operably coupled with a jaw drive sub-assembly 400 of actuation assembly 100 (FIGS. 2A-2B) to enable selective actuation of end effector assembly 40 to grasp tissue “T” (FIGS. 8 and 10) therebetween and apply a jaw force within an appropriate jaw force range, as detailed below.

Tissue-contacting surfaces 46, 48 of jaw members 42, 44, respectively, are at least partially formed from an electrically conductive material and are energizable to different potentials to enable the conduction of RF electrical energy through tissue “T” (FIGS. 8 and 10) grasped therebetween, although tissue-contacting surfaces 46, 48 may alternatively be configured to supply any suitable energy, e.g., thermal, microwave, light, ultrasonic, ultrasound, etc., through tissue “T” (FIGS. 8 and 10) grasped therebetween for energy-based tissue treatment. Instrument 10 defines a conductive pathway (not shown) through housing 20 and shaft 30 to end effector assembly 40 that may include lead wires, contacts, and/or electrically-conductive components to enable electrical connection of tissue-contacting surfaces 46, 48 of jaw members 42, 44, respectively, to an energy source (not shown), e.g., an electrosurgical generator, for supplying energy to tissue-contacting surfaces 46, 48 to treat, e.g., seal, tissue “T” (FIGS. 8 and 10) grasped between tissue-contacting surfaces 46, 48.

With additional reference to FIGS. 2A-3, as noted above, actuation assembly 100 is disposed within housing 20 and includes an articulation sub-assembly 200, a knife drive sub-assembly 300, and a jaw drive sub-assembly 400. Articulation sub-assembly 200 is operably coupled between first and second input couplers 110, 120, respectively, of actuation assembly 100 and articulation cables 38 (FIG. 1A) such that, upon receipt of appropriate inputs into first and/or second input couplers 110, 120, articulation sub-assembly 200 manipulates cables 38 (FIG. 1A) to articulate end effector assembly 40 in a desired direction, e.g., to pitch and/or yaw end effector assembly 40.

Knife drive sub-assembly 300 is operably coupled between third input coupler 130 of actuation assembly 100 and the knife tube such that, upon receipt of appropriate input into third input coupler 130, knife drive sub-assembly 300 manipulates the knife tube to reciprocate the knife blade 315 between jaw members 42, 44 to cut tissue “T” (FIGS. 8 and 10) grasped between tissue-contacting surfaces 46, 48.

Jaw drive sub-assembly 400, as detailed below, is operably coupled between fourth input coupler 140 of actuation assembly 100 and drive rod 484 such that, upon receipt of appropriate input into fourth input coupler 140, jaw drive sub-assembly 400 pivots jaw members 42, 44 between the spaced-apart and approximated positions to grasp tissue “T” (FIGS. 8 and 10) therebetween and apply a jaw force within an appropriate jaw force range.

Actuation assembly 100 is configured to operably interface with a robotic surgical system 2000 (FIG. 4) when instrument 10 is mounted on robotic surgical system 2000 (FIG. 4), to enable robotic operation of actuation assembly 100 to provide the above-detailed functionality. That is, robotic surgical system 2000 (FIG. 4) selectively provides inputs, e.g., rotational inputs to input couplers 110-140 of actuation assembly 100 to articulate end effector assembly 40, grasp tissue “T” (FIGS. 8 and 10) between jaw members 42, 44, and/or cut tissue “T” (FIGS. 8 and 10) grasped between jaw members 42, 44. However, it is also contemplated that actuation assembly 100 be configured to interface with any other suitable surgical system, e.g., a manual surgical handle, a powered surgical handle, etc. For the purposes herein, robotic surgical system 2000 (FIG. 4) is generally described.

Turning to FIG. 4, robotic surgical system 2000 is configured for use in accordance with the present disclosure. Aspects and features of robotic surgical system 2000 not germane to the understanding of the present disclosure are omitted to avoid obscuring the aspects and features of the present disclosure in unnecessary detail.

Robotic surgical system 2000 generally includes a plurality of robot arms 2002, 2003; a control device 2004; and an operating console 2005 coupled with control device 2004. Operating console 2005 may include a display device 2006, which may be set up in particular to display three-dimensional images; and manual input devices 2007, 2008, by means of which a person, e.g., a surgeon, may be able to telemanipulate robot arms 2002, 2003 in a first operating mode. Robotic surgical system 2000 may be configured for use on a patient 2013 lying on a patient table 2012 to be treated in a minimally invasive manner. Robotic surgical system 2000 may further include a database 2014, in particular coupled to control device 2004, in which are stored, for example, pre-operative data from patient 2013 and/or anatomical atlases.

Each of the robot arms 2002, 2003 may include a plurality of members, which are connected through joints, and a mounted device which may be, for example, a surgical tool “ST.” One or more of the surgical tools “ST” may be instrument 10 (FIG. 1A), thus providing such functionality on a robotic surgical system 2000.

Robot arms 2002, 2003 may be driven by electric drives, e.g., motors, connected to control device 2004. The motors, for example, may be rotational drive motors configured to provide rotational inputs, e.g., to selectively rotationally drive input couplers 110-140 (FIG. 2B) of surgical instrument (FIG. 1A) to accomplish a desired task or tasks. Control device 2004, e.g., a computer, may be configured to activate the motors, in particular by means of a computer program, in such a way that robot arms 2002, 2003, and, thus, their mounted surgical tools “ST” execute a desired movement and/or function according to a corresponding input from manual input devices 2007, 2008, respectively. Control device 2004 may also be configured in such a way that it regulates the movement of robot arms 2002, 2003 and/or of the motors.

Control device 2004, more specifically, may control one or more of the motors based on rotation, e.g., controlling to rotational position using a rotational position encoder (or Hall effect sensors or other suitable rotational position detectors) associated with the motor to determine a degree of rotation output from the motor and, thus, the degree of rotational input provided to the corresponding input coupler 110-140 (FIG. 2B) of surgical instrument 10 (FIG. 1A). Alternatively or additionally, control device 2004 may control one or more of the motors based on torque, current, or in any other suitable manner.

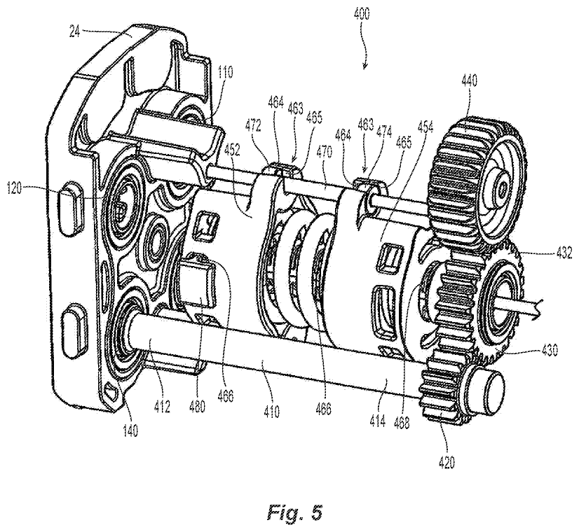

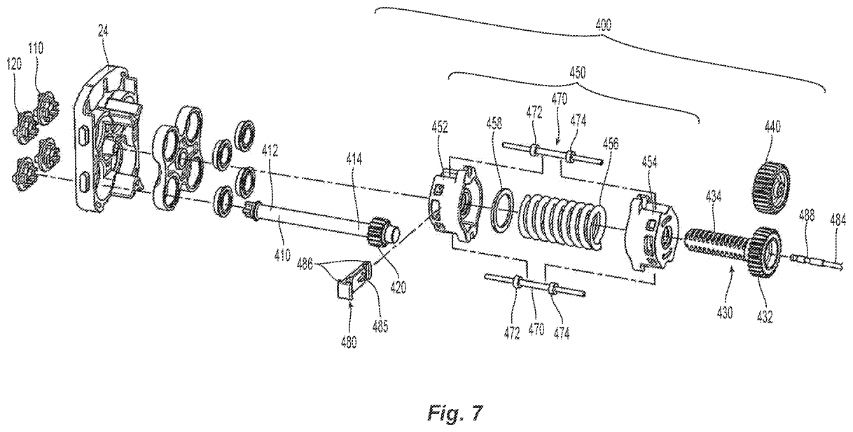

With reference to FIG. 5-7, jaw drive sub-assembly 400 of actuation assembly 100 is shown generally including an input shaft 410, an input gear 420, a drive gear 430, a thumbwheel 440, a spring force assembly 450, and a drive rod assembly 480.

Input shaft 410 includes a proximal end portion 412 operably coupled to fourth input coupler 140 and a distal end portion 414 having input gear 420 engaged thereon such that rotational input provided to fourth input coupler 140 drives rotation of input shaft 410 to, thereby, drive rotation of input gear 420. Input gear 420 is disposed in meshed engagement with round gear 432 of drive gear 430 such that rotation of input gear 420, e.g., in response to a rotational input provided at fourth input coupler 140, effects rotation of drive gear 430 in an opposite direction. Thumbwheel 440 is also disposed in meshed engagement with round gear 432 of drive gear 430 such that rotation of thumbwheel 440 effects rotation of drive gear 430 in an opposite direction, thus enabling manual driving of drive gear 430 via manipulation of thumbwheel 440. Drive gear 430, in addition to round gear 432, further includes a lead screw 434 fixedly engaged, e.g., monolithically formed, with round gear 432 such that rotation of round gear 432 effects similar rotation of lead screw 434.

Spring force assembly 450 includes a proximal hub 452, a distal hub 454, a compression spring 456, and a spring washer 458, although suitable force-limiting assemblies are also contemplated such as, for example, utilizing a torsion spring, a compliant feature, etc. Spring force assembly 450 further includes a pair of guide bars 470.

Proximal and distal hubs 452, 454 of spring force assembly 450 may be identical components that are oriented, positioned, and/or coupled to other components differently, thus providing different functionality while reducing the number of different parts required to be manufactured. The features of proximal and distal hubs 452, 454 are detailed below to the extent necessary to facilitate understanding of the present disclosure and, thus, although some features may be detailed with respect to only one of the proximal or distal hub 452, 454 and the function associated therewith, similar features may be provided on the other of the proximal or distal hub 452, 454 without the associated function. Alternatively, proximal and distal hubs 452, 454 may be manufactured as different components.

Proximal and distal hubs 452, 454 of spring force assembly 450 each include a retainer guide 463 extending radially outwardly from opposed sides thereof. Each retainer guide 463 defines a trough 464 and includes a shoulder 465 extending into the respective trough 464. Proximal and distal hubs 452, 454 are oppositely-oriented relative to one another such that the open ends of the cavities defined therein face one another and such that the shoulder 465 of each pair of retainer guides 463 of proximal and distal hubs 452, 454 face away from one another.

Proximal hub 452 further includes a transverse slot 466 defined therethrough that is configured to receive lock plate 482 of drive rod assembly 480 to fix lock plate 482 and, thus, a proximal end portion of drive rod 484 relative to proximal hub 452 (see FIGS. 9 and 11). Once engaged in this manner, drive rod 484 is locked in position coaxially disposed through proximal hub 452, distal hub 454, compression spring 456, and drive gear 430.

Distal hub 454 defines a threaded central bore 468 extending therethrough. Threaded central bore 468 receives lead screw 434 of drive gear 430 therethrough in threaded engagement therewith such that rotation of lead screw 434 drives translation of distal hub 454 longitudinally along lead screw 434.

Compression spring 456 is disposed between proximal and distal hubs 452, 454 with a proximal portion thereof disposed within the cavity defined within proximal hub 452 and a distal portion thereof disposed within the cavity defined within distal hub 462. At least a portion of compression spring 456 is disposed about and/or configured to receive a portion of lead screw 434 of drive gear 430 therethrough. Spring washer 458 is positioned within the cavity of proximal hub 452 between proximal hub 452 and compression spring 456, although other configurations are also contemplated.

Each guide bar 470 is slidably received within the troughs 464 of the corresponding pair of retainer guides 463 of proximal and distal hubs 452, 454. Each guide bar 470 includes a pair of spaced-apart rims 472, 474 engaged thereon that are configured to abut shoulders 465 of the respective retainer guides 463, thereby defining a maximum distance between proximal and distal hubs 452, 454. However, proximal and/or distal hubs 452, 454 are permitted to slide along guide bars 470 towards one another, as detailed below.

Continuing with reference to FIGS. 5-7, drive rod assembly 480 includes lock plate 482 and drive rod 484. Lock plate 482 defines a central keyhole 485 and a pair of slots 486, e.g., arcuate slots, defined on a distal face of lock plate 482 on either side of central keyhole 485. Lock plate 482 is configured for insertion through transverse slot 466 of proximal hub 452 and, once installed therein, portions of spring washer 458 are configured for receipt within slots 486 to secure lock plate 482 in engagement within proximal hub 452. Spring washer 458 is maintained in position within slots 486 under the bias of compression spring 456 which, at the maximum distance between proximal and distal hubs 452, 454 (as set by rims 472, 474 of guide bars 470 and shoulders 465 of retainer guides 463), is pre-compressed.

Drive rod 484, as noted above, includes a distal end portion operably coupled to cam-slot assembly 52 of end effector assembly 40 (FIG. 1). Drive rod 484 extends proximally through shaft 30, housing 20, and actuation assembly 100 (see FIGS. 1A, 2A-3) and is engaged within lock plate 482 at a proximal end portion of drive rod 484. More specifically, drive rod 484 defines a waist 488 towards the proximal end thereof that is configured to lock in engagement within central keyhole 485 of lock plate 482, e.g., via longitudinal translation of drive rod 484 into central keyhole 485 until waist 488 is aligned with central keyhole 485, followed by transverse movement of drive rod 484 relative to lock plate 482, to thereby fix the proximal end portion of drive rod 484 relative to lock plate 482 and, thus, relative to proximal hub 452 due to the engagement of lock plate 482 within proximal hub 452.

Referring to FIGS. 8-11, in use, jaw members 42, 44 are initially disposed in the spaced-apart position (FIG. 8) and, correspondingly, proximal and distal hubs 452, 454 are disposed in a distal-most position such drive rod 484 is disposed in a distal-most position (FIG. 9). Further, in this position, compression spring 456 is disposed in a least-compressed condition; although, as noted above, even in the least-compressed condition, compression spring 456 is partially compressed due to the retention of compression spring 456 in a pre-compressed configuration between proximal and distal hubs 452, 454.

In response to an input to close end effector assembly 40, e.g., rotational input by the corresponding motor of robotic surgical system 2000 (FIG. 4) to fourth input coupler 140 (FIGS. 5-7), drive shaft 410 is rotated to thereby rotate input gear 420 which, in turn, rotates drive gear 430 such that distal hub 454 is translated proximally towards proximal hub 452 (see FIG. 9). Proximal translation of distal hub 454 urges distal hub 454 against compression spring 456. Initially, where forces resisting approximation of jaw members 42, 44 are below a threshold corresponding to the spring value of compression spring 456, the jaw force applied by jaw members 42, 44 is relatively low such that the urging of distal hub 454 proximally against compression spring 456 urges compression spring 456 proximally which, in turn, urges lock plate 482 and, thus, drive rod 484 proximally to pivot jaw member 42 relative to jaw member 44 from the spaced-apart position towards the approximated position to grasp tissue “T” therebetween (FIGS. 8 and 10).

Upon further approximation of jaw members 42, 44 to grasp tissue “T” therebetween, the forces resisting approximation of jaw members 42, 44, e.g., tissue “T” resisting compression, may reach the threshold and, thus the jaw force applied by jaw members 42, 44 may reach a corresponding threshold. In order to maintain the jaw force applied by jaw members 42, 44 within a jaw force range such as, for example, from about 3 kg/cm² to about 16 kg/cm², application of further jaw force by jaw members 42, 44 is inhibited beyond the threshold point despite further rotational input to fourth input coupler 140 (FIGS. 5-7). More specifically, once the threshold has been reached, further rotational input to fourth input coupler 140 (FIGS. 5-7) rotates drive shaft 410, input gear 420, and drive gear 430 to translate distal hub 454 further proximally into compression spring 456. However, rather than compression spring 456 urging proximal hub 452 further proximally to continue approximation of jaw members 42, 44 and increase the closure force applied therebetween, compression spring 456 is compressed, enabling proximal hub 452 and, thus, drive rod 484 to remain in position, thus inhibiting application of additional jaw force between jaw members 42, 44 (see FIGS. 10 and 11).

With tissue “T” grasped between jaw members 42, 44 under an appropriate jaw force, energy may be supplied to jaw members 42, 44 to treat, e.g., seal tissue “T.” Thereafter, the knife blade 315 may be advanced between jaw members 42, 44 to cut the treated tissue “T,” e.g., by providing a rotational input to input coupler 130 (FIG. 6) to actuate knife drive sub-assembly 300 to translate the knife tube distally to thereby advance the knife blade 315 between jaw members 42, 44 to cut the treated tissue “T.” Alternatively, tissue “T” may be cut without first treating the tissue “T”and/or tissue “T”may be treated without subsequent cutting.

Once tissue “T” is cut, an opposite rotation input is provided to input coupler 130 (FIG. 6) to return the knife blade 315 to its initial position proximally of body portions 43b, 45b of jaw members 42, 44 (see FIG. 1A). Thereafter, an opposite input is provided to input coupler 140 (FIGS. 5-7) to return jaw members 42, 44 back towards the spaced-apart position to release the sealed and/or cut tissue.

Referring generally to FIGS. 1A, 2A-11, as detailed above, calibration information, setting information, use information, and adjustment information, among other information, are stored in the storage device of electronics 92 of instrument 10, in robotic surgical system 2000 (FIG. 4), and/or in other accessible storage devices. The calibration information may include an algorithm(s), set point(s), look-up table(s), machine learning program(s), and/or other information to enable determination of home/initial positions of the various components of instrument 10 such as, for example: the open position of jaw members 42, 44, the retracted position of the knife blade 315, the un-articulated configuration of shaft 30 and end effector assembly 40, etc.

The setting information may include, for example, jaw drive information, e.g., a degree of rotational input to input coupler 140 required to move jaw members 42, 44 from the open position towards the closed position to grasp tissue “T” between tissue-contacting surfaces 46, 48 and apply a jaw force or jaw force within a jaw force range thereto; knife deployment information, e.g., a degree of rotational input to input coupler 130 required to deploy the knife blade 315 from the retracted position to an extended position to cut tissue “T” between tissue-contacting surfaces 46, 48; and/or articulation control information, e.g., a degree of rotational input to input couplers 110 and/or 120 required to articulate end effector assembly 40 from the un-articulated position to one or more articulated positions (for example, a maximum positive yaw position, a maximum negative yaw position, a maximum positive pitch position, and a maximum negative pitch position); etc. The setting information may be determined based on testing during manufacturing (e.g., for each instrument, each unit of instruments, or for all instruments), may be determined via mathematical simulation, utilizing machine learning, using theoretical formulae, combinations thereof, etc.

The use information may include, for example, a number of connections to a robotic surgical system, elapsed time of use/connection, elapsed idle time, elapsed time of active use, age (time since manufacture), number of jaw member approximations, number of energy activations, number and/or manner of articulations, number of knife blade 315 deployments, etc. Robotic surgical system 2000 may write and/or update the use information stored in the storage device 92 of instrument 10 (and/or elsewhere) periodically, continuously, upon occurrence of an event, or in any other suitable manner.

Some or all of the setting information may be basis information that can be adjusted periodically, continuously, upon occurrence of certain events, and/or based on external inputs (user-provided input, sensor or other component feedback, etc.). For example, the basis setting information may be adjusted, e.g., at robotic surgical system 2000, based upon one or more current conditions of the instrument 10 and/or the current use information, as indicated by the adjustment information. The adjustment information for each corresponding setting may include an algorithm(s), set point(s), look-up table(s), machine learning program(s), etc. The adjustment information may be determined experimentally, via mathematical simulation, utilizing machine learning, using theoretical formulae, combinations thereof, etc.

By way of example, the jaw drive setting information may provide basis information indicating that “X” degrees of rotational input to input coupler 140 is required to move jaw members 42, 44 from the open position towards the closed position to grasp tissue “T” between tissue-contacting surfaces 46, 48 and apply a jaw force or jaw force within a jaw force range thereto. Thus, in the absence of modification to this jaw drive setting information, upon receiving a signal to approximate jaw members 42, 44 to grasp tissue between tissue-contacting surfaces 46, 48 for tissue treatment, e.g., sealing, control device 2004 controls the appropriate motor(s) of robotic surgical system 2000 to impart “X” degrees of rotational input to input coupler 140 such that tissue-contacting surfaces 46, 48 grasp tissue “T” therebetween under the applied jaw force or jaw force within the jaw force range.

However, it has been found that the jaw force or jaw force range applied in response to input of a set degree of rotational input to input coupler 140 may vary over the usable life of instrument 10 and/or based upon a current condition of instrument 10, e.g., whether end effector assembly 40 is disposed in an un-articulated position, partially articulated position, or fully articulated position. The stage of useable life of instrument 10 may be determined based upon some or all of the above-noted use information and may affect the jaw force or jaw force range due to, for example, changes in component stiffness/elasticity, establishment of “memory” positions of components/connections, changes in force transmission across joints/connections, changes in tolerances, changes in frictional loss, component wear, component and/or joint/connection degradation, etc. The current condition of instrument 10 may be determined by control device 1004 and/or other components of robotic surgical system 2000 based upon feedback data, previous inputs, visual or other tracking information, etc., and may affect the jaw force or jaw force range due to actuation force changes, actuation distance changes, friction changes, etc.

In order to account for the above changes, the adjustment information enables adjustment of the basis jaw drive setting, e.g., “X” degrees, to an adjusted jaw drive setting, e.g., “Y” degrees, based upon the use and/or current condition of instrument 10 using the algorithm(s), set point(s), look-up table(s), machine learning program(s), etc. As such, with the adjusted jaw drive setting information implemented, upon receiving a signal to approximate jaw members 42, 44 to grasp tissue between tissue-contacting surfaces 46, 48 for tissue treatment, e.g., sealing, control device 1004 controls the appropriate motor(s) of robotic surgical system 2000 to impart “Y” degrees of rotational input to input coupler 140 such that tissue-contacting surfaces 46, 48 grasp tissue “T” therebetween under the applied jaw force or jaw force within the jaw force range. Thus, the same jaw force or jaw force range is achieved despite changing input requirements.

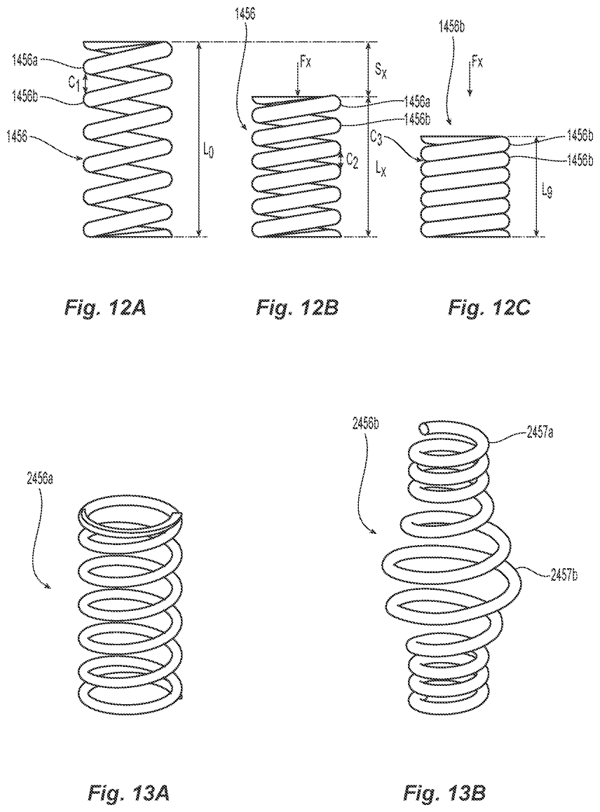

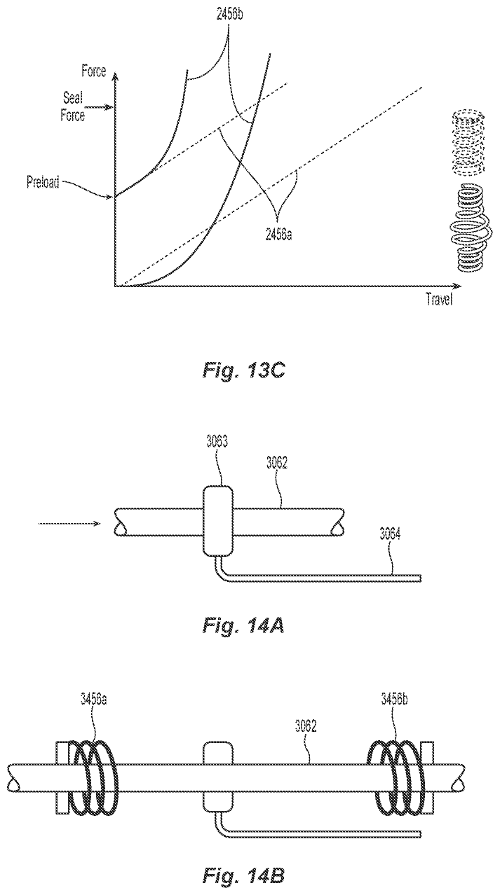

The present disclosure, however, is not limited to adjusting jaw drive setting information for applying jaw force but, rather, may apply to adjustment of any other suitable setting information, e.g., knife deployment information, articulation control information, etc. Further, the present disclose is not limited to instrument 10 but may also apply to any other suitable surgical instrument. Indeed, the methods provided in accordance with the present disclosure and detailed below with reference to FIGS. 12 and 13 may be utilized with instrument 10 for adjusting jaw drive setting information or may be utilized with any other suitable instrument and/or desired manipulation thereof.

Turning now to FIGS. 12A-14B, various embodiments of present disclosure utilize one or more springs with the sealing device to vary, alter, and/or regulate pressure loads between the jaw members 42, 44 during sealing or, in some instances, for safety measures. FIG. 12A, for example, shows a spring 1456 for use with the drive assembly 450 of FIG. 1A wherein the spring 1456 is compressible between a first length Lo to a fully compressed length Lg under a force Fₓ. It is important to note that other mechanisms of the instrument 10 that employ a spring may be configured to take advantage of the features described with respect to FIGS. 12A-14B, e.g., knife spring, return springs, etc.

More particularly, in an initial state with the jaw members 42, 44 fully open, spring 1456 is disposed in a fully expanded state as shown in FIG. 12A (although it is envisioned that spring 1456 may be preloaded under slight compression to reduce “slop”). In this state, adjacent coils 1456a, 1456b are spaced or “pitched” a distance C₁ relative to one another under the spring's compression rate or “spring rate” k. Upon actuation of the input coupler 140, the spring 1456 is compressed an initial distance such that the coils 1456a, 1456b are pitched to a distance C₂ under a force Fₓ as described above to close the jaw members 42, 44 about tissue.

FIG. 12C shows the spring 1456 under full compression (e.g., solid length) wherein the coils 1456a, 1456b are essentially touching or “coil-on-coil”. It is envisioned that the coil-on-coil position of the spring 1456 may be utilized for additional purposes, e.g., to provide a direct or a solid drive force from the motor (or handle) to the jaw members 42, 44 without spring assistance or, in other embodiments, provide feedback to the robotic system 2000, e.g., safety information such as over-compression of tissue warning. More particularly, spring 1456 may be configured such that some percentage of travel of the input coupler, e.g., 60%, will yield a force Fₓ on the tissue offset by the spring rate of spring 1456. When the spring 1456 is fully compressed to a coil-on-coil configuration as illustrated in FIG. 12C, the spring 1456 essentially becomes solid structure translating the force Fₓ from drive assembly 450 and input coupler 140 directly the jaw members 42, 44 and on the tissue. As a result, the force on the tissue may be varied over the course of travel of the drive assembly 450. As can be appreciated, with hand-held instruments, this is particularly advantageous, i.e., initial handle movement for manipulation and handling of tissue under slight compression to full compression when sealing tissue. In embodiments, the spring 1456 may be configured to offset from about 30% to about 80% of the forces associated with the input control on the drive assembly 450 or drive rod 484 depending upon a particular purpose.

As mentioned above, the coil-on-coil configuration of the spring 1456 illustrated in FIG. 12C may be utilized to alert the surgeon if the tissue is being over compressed. In other words, a sensor (not shown) may be employed to alert the surgeon that the spring coils 1456a, 1456b are touching such that further drive input from coupler 140 (or further handle compression of a hand-held instrument of FIG. 1B) may result in over compression of the tissue. Alternatively, the coil-to-coil contact of the spring coils 1456a, 1456b may simply alert the user to the change in compression force on the tissue, e.g., from controlled by the spring rate of the spring 1456 to direct input force from coupler 140 (or handle 1030).

FIGS. 13A-13C show another embodiment of the present disclosure wherein the spring may have a variable or progressive spring rate over the course of compression (or extension). More particularly, spring 2456a of FIG. 13A is a linear spring wherein the spring rate k over the travel (i.e., compression or extension) is constant or at a linear rate as shown in the graph of FIG. 13C. A variable or progressive spring 2456b (FIG. 13B) includes coils 2457a, 2457b that vary in diameter which, in turn, vary the spring rate k over the course of travel. The graph of FIG. 13C highlights the difference between a linear spring 2456a and the progressive spring 2456b. The diameter of the progressive spring 2456b at various points along the progressive spring 2456b may be mathematically correlated to the spring rate k thereof.

The variable spring rate k of progressive spring 2456b is configured to offload forces associated with the drive rod 484 during actuation thereof; the spring including a first spring rate “k₁” associated with initial activation thereof and one or more different spring rates “k₂” as the spring is compressed. Put simply, upon initial actuation of the drive rod 484, the spring rate k may be less facilitating gripping and manipulating tissue. As the spring 2456b is compressed, the spring rate k increases towards a seal force (see FIG. 13C). As such, the compression force Fₓ on the tissue will vary over the course of travel of the spring 2456b, i.e., the rate of force increases getting greater as the spring 2456b is compressed.

In other embodiments, one or more springs may be utilized to alert the surgeon that a higher than anticipated force has been exerted on a particular part of the instrument, e.g., shaft, knife blade, etc. or tissue via the jaw members 42, 44. More particularly, and similar to a fishing pole, a spring torque wrench (not shown) may be employed to prevent over torque on one or instrument parts, e.g., drive rod 484. When the maximum allowable torque on the drive rod 484 is reached (which is equivalent to a maximum allowable closure pressure), any additional torque on the drive rod 484 is dissipated by the spring torque wrench. Alternatively, and with input coupler 140, when the maximum allowable torque on the drive rod 484 is reached, a switch may be activated to cut off further rotational input from coupler 140 or an alert may be signaled to the surgeon to release stop activation thereof, or a spring torque wrench may also be utilized. As can be appreciated, this prevents over-compression of the tissue during sealing (or handling) and/or may be configured to prevent bending or breaking of the drive rod 484 or other driven components.

In embodiments, the spring torque wrench may be applied to the articulation sub-assembly 200 to prevent over-articulation of the distal segment 32 of shaft 30 or end effector 40 which can overstress other internal components. Moreover, the spring torque wrench may be utilized with the knife tube 62 to prevent damage to the knife 315 when the knife 315 encounters overly thick tissue, bone or a staple.

Springs may be configured to prevent damage to other component features, e.g., the knife 315. FIG. 14B shows an example of a pair of springs 3456a, 3456b disposed on either end of the knife tube 3062 which are configured to offset the force Fₓ when applying a preset or preconfigured tolerance. A load cell 3063 (FIG. 14A, FIG. 14B) may be disposed atop the knife tube 3062 and configured to measure the strain (or stress) applied thereto during actuation of the knife 315 (e.g., translation of the knife tube 3062). A load cell 3063 connects to a measurement device, switch, alarm or robotic surgical system 2000 to signal the user regarding the strain (or stress) on the knife tube 3062. Springs 3456a, 3456b may be configured to offload the strain (or stress) on the knife tube 3062 once a threshold is reached either during actuation (e.g., translation of the knife tube 3062) or articulation of the knife tube 3062 (bending). Spring 3456b may also be utilized to handle imperfect length changes along the knife tube 3062.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

It will be understood that various modifications may be made to the aspects and features disclosed herein. Therefore, the above description should not be construed as limiting, but merely as exemplifications of various aspects and features. Those skilled in the art will envision other modifications within the scope and spirit of the claims appended thereto.

What is claimed is:

1. A surgical instrument, comprising:
a housing including a shaft extending therefrom having an end effector at a distal end thereof, the end effector including first and second jaw members, at least one of the jaw members moveable between a spaced apart position relative to the other jaw member and a closed position wherein the jaw members cooperate to grasp tissue therebetween; and
a drive assembly including a drive rod operably associated with the at least one jaw member and actuatable to move the at least one jaw member between the spaced apart position and the closed position, and a spring having a spring rate “k” operably associated with the drive rod and configured to offload forces associated therewith during actuation of the drive rod, the spring including at least a first length wherein adjacent coils of the spring are spaced a distance relative to one another such that the spring offloads forces associated with the drive rod as per the spring rate “k” of the spring and a fully compressed length wherein the adjacent coils of the spring abut one another and the forces associated with the drive rod are transferred through the spring.

2. The surgical instrument according to claim 1, wherein the spring is configured to offload a certain percentage of forces associated to the drive rod prior to reaching a fully compressed length.

3. The surgical instrument according to claim 2, wherein the percentage of forces offloaded to the drive rod is in the range of about 30% to about 80%.

4. The surgical instrument according to claim 1, wherein in the fully compressed length forces from an input operably associated with the drive assembly are transferred directly to the drive rod.

5. The surgical instrument according to claim 4, wherein the input operably associated with the drive assembly includes an input coupler from a robotic surgical system.

6. The surgical instrument according to claim 4, wherein the input operably associated with the drive assembly includes a movable handle extending from the housing.

7. A surgical instrument, comprising:
a housing including a shaft extending therefrom having an end effector at a distal end thereof, the end effector including first and second jaw members, at least one of the jaw members moveable between a spaced apart configuration relative to the other jaw member and a closed position relative wherein the jaw members cooperate to grasp tissue therebetween; and
a drive assembly including:
a drive rod operably associated with the at least one jaw member and actuatable to move the at least one jaw member between the spaced apart position and the closed position; and
a progressive spring operably associated with the drive rod and having a variable spring rate “k” configured to offload forces associated with the drive rod during actuation thereof, the spring including a first spring rate “k₁” associated with initial activation thereof and at least one different spring rate “k₂” as the spring is compressed.

8. The surgical instrument according to claim 7, wherein the first spring rate “k₁” is less than the at least one different spring rate “k₂”.

9. The surgical instrument according to claim 7, wherein a diameter of the progressive spring varies along a length thereof.

10. The surgical instrument according to claim 9, wherein the spring rate of the progressive spring is directly proportional to the diameter thereof.

11. The surgical instrument according to claim 7, wherein the spring rate of the progressive spring is non-linear.

12. A surgical instrument, comprising:
a housing including a shaft extending therefrom having an end effector at a distal end thereof, the end effector including first and second jaw members, at least one of the jaw members moveable between a spaced apart configuration relative to the other jaw member and a closed position wherein the jaw members cooperate to grasp tissue therebetween;
a drive assembly including a drive rod operably associated with the at least one jaw member and actuatable to move the at least one jaw member between the spaced apart position and the closed position for grasping tissue;
a knife tube disposed within the shaft, the knife tube having a knife at a distal end thereof configured to cut tissue disposed between the jaw members; and
a spring having a spring rate “k” operably associated with the knife tube and configured to offload forces associated therewith during actuation of the knife tube, the spring including at least a first length wherein adjacent coils of the spring are spaced a distance relative to one another such that the spring offloads forces associated with the knife tube as per the spring rate “k” of the spring and a fully compressed length wherein the adjacent coils of the spring abut one another and the forces associated with the knife tube are transferred through the spring to prevent damage to the knife.

13. The surgical instrument according to claim 12, wherein the spring is configured to offload a certain percentage of forces associated to the knife tube prior to reaching a fully compressed length.

14. The surgical instrument according to claim 12, wherein an input coupler from a robotic surgical system is configured to actuate the knife tube.

15. The surgical instrument according to claim 12, wherein a trigger is operably associated with the knife tube and is configured to actuate the knife tube upon movement thereof.

16. The surgical instrument according to claim 12, further comprising a second spring operably associated with the knife tube, the second spring configured to offload forces associated with articulation of the shaft and the knife tube disposed therein.
